(19) United States
(12) Patent Application Publication
(10) Pub. No.: US 2003/0180355 A1
(43) Pub. Date: Sep. 25, 2003

(54) Combination therapy for hypertension

(76) Inventors: Amedeo Leonardi, Milan (IT); Abraham Sartani, Arese (IT); Giorgio Sironi, Pieve Emanuele (IT)

Correspondence Address: DARBY & DARBY P.C., Post Office Box 5257, New York, NY, 10150-5257, US

(21) Appl. No.: 10/274,430

(22) Filed: Oct. 16, 2002

Related U.S. Application Data
(60) Provisional application No. 60/344,601, filed on Oct. 23, 2001.

(30) Foreign Application Priority Data
Oct. 16, 2001 (IT) MI 2001A 002136

(51) Int. Cl. A61K 38/05; A61K 31/455; A61K 31/4015; A61K 9/20

(52) U.S. Cl. 424/465; 514/355; 514/19; 514/423

(57) ABSTRACT

Disclosed are compositions and methods for treating hypertension comprising enalapril and lercanidipine in amounts effective in combination to reduce blood pressure to a patent in need of treatment.

### RELATED APPLICATIONS

The present application claims priority under 35 U.S.C. 119 (e) of U.S. provisional application 60/344,601, filed Oct. 23, 2001 and priority under 35 U.S.C. 119 (a)-(d) of Italian patent applications MI 2001A 012136 filed Oct. 16, 2001. Each of the aforementioned applications is hereby incorporated herein by reference in its entirety.

### FIELD OF THE INVENTION

The present invention contemplates a method for treating hypertension with a combination of enalapril and lercanidipine.

### BACKGROUND OF THE INVENTION

Hypertension is one of the most common cardiovascular disease states. In the United States, over 50 million people have been diagnosed with hypertension (which is defined as a blood pressure greater than or equal to 140/90 mm Hg). Elevated arterial pressure can cause pathological changes in the vasculature and hypertrophy of the left ventricle. Due to the damage that can be produced by hypertension, it is proposed to be the principal cause of stroke, myocardial infarction, and sudden cardiac death. Additionally, it is believed to be a major contributor to cardiac failure, renal insufficiency, and dissecting aneurysm of the aorta.

The renin-angiotensin system is an important regulator of arterial pressure. The inactive angiotensinogen peptide is converted to the pro-peptide angiotensin I by the enzyme renin. Angiotensin I then is converted to the active angiotensin II form by the angiotensin converting enzyme. Angiotensin II then acts through a variety of receptor mediated mechanisms, such as increasing the total peripheral resistance and inhibiting the excretion of sodium and water by the kidneys, to increase arterial pressure.

Angiotensin converting enzyme (ACE) inhibitors are active agents that prevent the conversion of angiotensin I into angiotensin II. The hypotensive action of these active agents is well documented and such active agents have been used extensively in the treatment of hypertension. Examples of ACE inhibitors are described in U.S. Pat. Nos. 4,350,633; 4,344,949; 4,294,832; and 4,350,704.

Enalapril ((S)-1-[N-1-(ethoxycarbonyl)-3-phenylpropyl]-L-alanyl-L-proline) is a prodrug of the active ACE inhibitor, enalaprilat. Enalapril is rapidly absorbed following oral administration with peak plasma levels of enalaprilat occurring in about 3 to 4 hours. The antihypertensive action of enalapril is believed to result primarily from the suppression of the renin-angiotensin system as a result of inhibition of angiotensin II formation. The recommended starting dose of enalapril as monotherapy is 5 mg daily, with drug titration 10 to 40 mg per day. The most common dosage is 20 mg per day. Several weeks of therapy may be required to achieve optimal blood pressure reduction for a patient. Enalapril is commercially available from a variety of pharmaceutical suppliers (e.g., Merck (sold under the trade name Vasotec)) and has been approved for treatment of hypertension in several countries. Enalapril has various side-effects including headache, dizziness, fatigue, cough, gastrointestinal disturbance, sore throat, fever, diarrhea, loss of taste sensation, muscle cramps, unusual bruising, and edema.

Another class of active agents that is used for the treatment of hypertension is calcium antagonists. These active agents influence the influx of calcium ions into cells, especially smooth muscle cells. Inhibition of calcium influx produces a relaxation of smooth muscles, such as those around the arteries and veins, which leads to a decrease in the observed hypertension. Such active agents as well as their hypotensive activity are described in a number of publications and patent applications.

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist with long duration of action and high vascular selectivity. Its mechanism of antihypertensive activity is due to a direct relaxant effect on vascular smooth muscle, thus lowering total peripheral resistance. The recommended starting dose of lercanidipine as monotherapy is 10 mg daily by oral route, with a drug titration to 20 mg daily. Lercanidipine is rapidly absorbed following oral administration with peak plasma levels occurring 2-3 hours following dosing. Elimination is essentially via the hepatic route. By virtue of its high lipophilicity and high membrane coefficient, lercanidipine combines a short plasma half life with a long duration of action. In fact, the preferential distribution of the drug into membranes of smooth muscle cells results in membrane-controlled pharmacokinetics which is characterized by a prolonged pharmacological effect. In comparison to other calcium antagonists, lercanidipine is characterized by gradual onset and long-lasting duration of action despite decreasing plasma levels. In vitro studies show that isolated rat aorta response to high K⁺ may be attenuated by lercanidipine, even after the drug has been removed for 6 hours. Lercanidipine is commercially available from Recordati S.p.A. (Milan, Italy) and has been described along with methods for making it and resolving it into individual enantiomers in U.S. Pat. Nos. 4,705,797; 5,767,136; 4,968,832; and 5,696,139.

Clinical studies have shown that lercanidipine 10 mg daily (titrated to 20 mg daily in patients not responding or responding inadequately) provides a sustained pharmacological action and a significant antihypertensive effect. In hypertensive patients the onset of lercanidipine action is gradual and the drug has a consistent and sustained blood pressure lowering effect throughout the dosage interval. The gradual and smooth antihypertensive effect has been recently confirmed also by using the “Smoothness Index”, as described in Omboni and Zanchetti, Hypertension, 1998, 16:1831-8. The analysis of a large population of hypertensive patients has documented that lercanidipine is a very well tolerated drug devoid of major side effects. During this market experience no alarming signals on safety or drug-interactions emerged, indicating that the use in hypertensive patients may be considered safe. In man, lercanidipine is contraindicated (as all dihydropyridines) in patients with unstable angina or recent (<1 month) myocardial infarction.

Several pharmacological rationales have been used for combining an ACE-inhibitor and a calcium antagonist to treat hypertension. For example, the fact that multiple physiologic systems participate in blood pressure control has been proposed as the reason why individual active agents decrease in efficacy over time. The pharmacological intervention on one of these systems, is believed to trigger counter regulatory mechanisms. A combination of treatments increases the number of mechanisms potentially capable of reducing an elevated blood pressure and reduces the rate and magnitude of the adverse events produced by each drug. Further, the addition of one agent may counteract some deleterious effect of the other. Therefore a low-dose combination of two different agents reduces the risk of dose-related adverse reaction while still allowing sufficient blood pressure reduction. Optimal associations are those between a thiazide diuretic and an ACE-inhibitor or a calcium antagonist and an ACE-inhibitor. Associations between a calcium antagonist and a diuretic or between an ACE-inhibitor and a beta-blocker also can be used, but partial overlap of their mechanism of action may make their effectiveness less than the sum of individual agents (Mancia and Grassi, High Blood Pressure 1994; 3 (Suppl.4): 5-7).

The ACE-inhibitors attenuate vasocostriction through reduction of the vasocostrictive effect of angiotensin II and augmentation of the vasodilatatory kinins, whereas the calcium antagonists act through attenuating the transmembrane flux of calcium inhibit calcium-mediated electromechanical coupling in contractive tissue in response to numerous stimuli. Moreover, both classes of drugs facilitate salt and water excretion by the kidney through different mechanisms. The ACE-inhibitor restores the renal-adrenal response to salt loading, whereas the calcium antagonist possesses intrinsic natriuretic properties, probably through mechanism of inhibiting tubular salt and water reabsorption (Weir, AJH 1998; 11:163S-169S).

ACE-inhibitors also may reduce the counterregulatory effects induced by calcium antagonists (i.e. stimulation of the sympathetic system) and on the other hand the negative sodium balance induced by calcium antagonists could potentiate the hypotensive effects of ACE-inhibitors (Menard and Bellet J. Cardiovasc. Pharmacol 1993; 21 (Suppl.2):S49-S54).

In addition to pharmacological advantages, combination therapy has been requested to meet evolving guidelines that look for more aggressive treatment of blood pressure. For example, recent World Health Guidelines recommend a diastolic blood pressure lower than 85 mm Hg and a systolic blood pressure lower than 130 mm Hg in younger patients and in diabetic patients.

Currently, there are various fixed combinations of ACE-inhibitors and calcium antagonists that are marketed in Europe and in the United States. These include combinations of ramipril and felodipine, trandolapril and verapamil, enalapril and felodipine, benazepril and amlodipine, and enalapril and diltiazem. Many patients may experience side effects due to one or both of the administered active agents, or due to the specific combination of the two active agents. However, fixed combinations offer the possibility of administering a combination of active agents in a single dosage form. Such a form will likely increase the patient compliance. That is, such a dosage form will likely increase a patient's adhesion to a therapeutic scheme and will increase the success of such the treatment therapy.

Additionally, a number of patents are either nonresponsive to one or more of the available monotherapies, and some patients are not responsive to the aforementioned prior art combination therapies. There is no way at present to predict whether these patients will be responsive to therapy using a new combination of active ingredients. It has been calculated that, overall, 30-50% of patients are non-responders to monotherapy (this average does not include data of patients taking lercanidipine).

Therefore, there is a continuing need for effective combination anti-hypertensive treatments that have a long lasting, selective mechanism of action with few side effects.

### BRIEF DESCRIPTION OF THE DRAWINGS

FIG. 1. Graphical representation of the effect of placebo, lercanidipine (10 μg/kg) treatment, enalapril (100 μg/kg) treatment, and the combination of lercanidipine and enalapril on diastolic blood pressure in uninephroctomized anesthetized rats.

FIG. 2. Graphical representation of the effect of placebo, lercanidipine (10 μg/kg) treatment, enalapril (100 μg/kg) treatment, and the combination of lercanidipine and enalapril on systolic blood pressure in uninephroctomized anesthetized rats.

FIG. 3. Graphical representation of the effect of placebo, lercanidipine treatment, enalapril treatment, and the combination of lercanidipine and enalapril on diastolic blood pressure in renal hypertensive dogs.

FIG. 4. Graphical representation of the effect of placebo, lercanidipine treatment, enalapril treatment, and the combination of lercanidipine and enalapril on systolic blood pressure in renal hypertensive dogs.

FIG. 5. Categorization of patients based on prior (background) hypertension treatment. Candesartan: (±)-1-[[(cyclohexyloxy)carbonyl]oxy)ethyl 2-ethoxy-1-[[2′-(1H-tetrazol-5-yl) [1,1′-biphenyl]-4-yl] methyl-1H-benzimidazole-7-carboxylate; Perindopril: (2S, 3aS, 7aS)-1-[(S)-N-[(S)-1-carboxybutyl]alanyl]hexahydro-2-indolinecarboxcylic acid, 1-ethyl ester; Cilazapril: (1S, 9S)-9(((1S)-1-(ethoxycarbonyl)-3-pheynlpropyl)amino)octahydro-10-oxo-6H-pyridazino(1,2-a)(1,2)diazepine-1-carboxcylic acid; Ramipril: (2S, 3aS, 6aS,-1-(S)-N-[(S)-1-carboxy-3-phenyl-propyl]alanyl]octa hydrocyclopenta[b]pyrrole-2-carboxylic acid, 1-ethyl ester; Lisinopril: (S)-1-[N²-(1-carboxy-3-phenylpropyl)-L-lysyl]-L-proline dihydrate; Losartan: 2-butyl-4-chloro-1-[-(O-1H-tetrazol-5-ylphenyl) benzyl]imidazole-5-methanol monopotassium salt.

FIG. 6. Graphical representation of the effect of addition of an amount of lercanidipine, for eight weeks, to patients currently taking different amounts of enalapril on sitting diastolic blood pressure.

FIG. 7. Graphical representation of the effect of addition of an amount of lercanidipine, for eight weeks, to patients currently taking different amounts of enalapril on sitting systolic blood pressure.

### SUMMARY OF THE INVENTION

The present invention contemplates methods for treating hypertension in four classes of patients. The first class of patients are those that are responders to monotherapy with either enalapril or lercanidipine, but who suffer from side-effects and for whom it would be desirable to decrease the dosage amount of the active agent used in monotherapy. In other words, these active agents produce antihypertensive activity and decrease the patient's blood pressure by the predetermined increment. A combination of enalapril and lercanidipine is particularly suitable for such patients.

Accordingly, in one aspect, the present invention is directed to a method for treating hypertension in a patient in need thereof, the method comprising administering to the patient a first amount of lercanidipine and a second amount of enalapril, where the amounts in combination are effective to reduce blood pressure in the patient by at least a predetermined increment and thus restore blood pressure to within acceptable limits; where at least one of the first amount and the second amount is either ineffective to produce a reduction in blood pressure in the patient, or the reduction in blood pressure is less than the predetermined increment. In other words, the amounts of the two agents employed in the combination would each be suboptimal or totally ineffective if administered as monotheraphy.

The second patient class are of patients who are “nonresponders” to monotherapy. In these patients, the active agent or agents alone do not produce anithypertensive activity. In another aspect, the present invention encompasses a method for treating hypertension in a nonresponder patient in need thereof, the method comprising administering to the patient a first amount of lercanidipine and a second amount of enalapril, where the amounts in combination are effective to reduce blood pressure in the patient by at least a predetermined increment, and thus restore blood pressure to within acceptable limits. The patient would usually have been previously determined not to respond or to respond insufficiently to monotherapy with lercanidipine or enalapril, or even with another single antihypertensive agent.

The third class of patients are of patients who are partial responders to monotherapy and combination therapy. Monotherapy or combination therapy produces an antihypertensive effect in these patients, but the therapy does not decrease the blood pressure by the predetermined increment. In another aspect, the present invention encompasses a method for treating hypertension in a partial responder patient in need thereof, the method comprising administering to the patient a first amount of lercanidipine and a second amount of enalapril, wherein the amounts in combination are effective to reduce blood pressure in the patient by at least a predetermined increment, wherein each of the first amount and the second amount if administred alone. is ineffective to produce a reduction in blood pressure by the predetermined increment.

The fourth class of patients includes those that are responders to monotherapy but have been previously determined (or are expected) to become nonresponders over time. Conventionally, patients in this class, upon becoming nonresponders, would then require a monotherapy involving higher dosage amounts of the same active agent or would need a change of medication to another active agent to treat hypertension (i.e., reduce blood pressure by the predetermined increment). However, it should be noted that these patients may not further respond to increased dosages due to maximal efficacy of the compound having been reached. The cause for such a change in a patient's response also may be a compensation (counterregulatory) mechanism or another cause.

In yet another aspect, the present invention encompasses a method for treating hypertension in a patient within the fourth class, where the patient has been previously determined to be responsive to monotherapy with lercanidipine or with enalapril, the method comprising administering to the patient a composition comprising a first “combination therapy amount” of lercanidipine and a second combination therapy amount of enalapril, where the combination therapy amounts are in combination effective to reduce the patient's blood pressure by at least the predetermined increment, and thus restore blood pressure to within acceptable limits.

In yet another aspect, the present invention encompasses methods of treating a patient within any of the aforementioned classes wherein said patient is a diabetic (e.g., a type II diabetic), although preferably a patient within any of the aforementioned classes is not a diabetic (e.g., is not a type II diabetic).

Lastly, in principle, the present invention can be employed with naive patients although the regulatory authorities guidelines do not encourage such a practice.

Compositions and dosage forms are further contemplated by the present invention.

### DETAILED DESCRIPTION OF PREFERRED EMBODIMENTS

As to used herein, the term “hypertension” refers to abnormally high arterial blood pressure, when compared to prior blood pressure readings, and the abnormally high value is maintained over a specified time period. Conventionally, the time period is 3-6 months. The increase may be observed in systolic pressure, diastolic pressure, or both. Conventionally, hypertension is defined as a blood pressure of equal to or greater than 140/90 mm Hg. Blood pressure may be measured by any method known in the art. Such methods include, but are not limited to direct arterial puncture, oscillometry, Doppler ultrasonography, and a sphygmomanometer. In a preferred embodiment, blood pressure is measured with a sphygmomanometer. While the person taking the measurement listens to the pulse of the patient and watches the sphygmomanometer gauge, two measurements (systolic pressure and diastolic pressure) are recorded. Blood pressure is measured in millimeters of mercury (mm Hg).

The terms “systolic” and “systolic pressure” refer to the pressure induced by the contraction of the heart by which the blood is forced onward and the circulation kept up. The terms “diastolic” and “diastolic pressure” refer to the pressure induced by the dilatation of the cavities of the heart during which they fill with blood. Typically, blood pressure is expressed as two numbers separated by a slash, where the first number is the systolic pressure and the second number is the diastolic pressure. As mentioned above, the pressure is conventionally expressed as mm Hg.

The term “antihypertensive activity” refers to the effect of an active agent to lower the blood pressure of a patient with hypertension. In one embodiment, the blood pressure is decreased by at least 20 mm Hg for systolic pressure or by at least 10 mm Hg for diastolic pressure. In another embodiment, the antihypertensive activity refers to the effect of an active agent to lower the blood pressure by at least 20 mm Hg for systolic pressure and by at least 10 mm Hg for diastolic pressure. The active agent may or may not decrease the blood pressure in a person that does not have hypertension or may not decrease blood pressure in all persons with hypertension. In a preferred embodiment, the active agent decreases a patient's blood pressure to below 140/90 mm Hg.

The term “active agent” or “active ingredient” refers to a compound that produces a pharmacological effect that leads to a physiological change. As used herein, the active agents are antihypertensive agents, such as lercanidipine and enalapril, which are employed in the combination treatment of the invention. Conventionally, an active agent is considered as having an antihypertensive effect if it decreases either systolic or diastolic blood pressure by at least 10 mm Hg.

The term “predetermined increment” refers to the minimum reduction in blood pressure that is needed for a patient to decrease blood pressure to or below 140/90. Thus, an active agent which at a dosage tolerated by the patient achieves reduction by a predetermined increment is considered effective to treat hypertension in the specific patient, and the patient is considered responsive to this agent (also known as a “responder”). In other words, if an active agent decreases blood pressure by a predetermined increment in one patient (i.e., has sufficient antihypertensive activity in the patient) but does not decrease blood pressure by the predetermined increment in another patient (i.e., does not have sufficient antihypertensive activity in the patient), then the first patient is responsive to the treatment (a “responder”, as defined below) but the second patient is not (a “nonresponder”, as defined below). The decrease in blood pressure can be in the systolic pressure, diastolic pressure, or both.

As used herein, the term “responder” refers to a patient that has previously responded to a treatment for hypertension involving administration of a particular active agent (or combination of active agents) in a particular amount or amounts. In other words, the active agent or active agents have “antihypertensive activity” and reduce the patient's blood pressure by the “predetermined increment”. A determination of responsiveness to an antihypertensive regimen may require administration of a particular agent in a particular amount and frequency for a period of time, usually 1 month for ACE inhibitors and calcium antagonists. Such treatments include, but are not limited to, administration of ACE inhibitors, calcium channel blockers, beta blockers, and diuretics. The phrase “responsive to monotherapy” refers to patients who are administered only one active agent (monotherapy) and the monotherapy achieves a reduction in blood pressure by the “predetermined increment” as that term is defined above. In a specific embodiment, the antihypertensive activity is defined as at least a decrease of 20 mm Hg in systolic pressure or as at least a decrease of 10 mm Hg for diastolic pressure.

The term “nonresponder” refers to a patient who has been determined not to have responded to treatment for hypertension with a particular agent or combination of agents, i.e., for whom the regimen has not achieved a reduction in blood pressure. In other words, the active agent or active agents do not have “antihypertensive activity” in the patient, and therefore the patient's blood pressure is not decreased by the “predetermined increment”. The term encompasses patients that do not undergo any decrease in blood pressure upon treatment e.g. with lercanidipine alone or enalapril alone.

The term “partial responder” refers to a patient for whom a particular active agent (or combination of active agents), in a particular amount or amounts, produces “antihypertensive activity” in the patient but does not decrease blood pressure by the “predetermined increment”. Increases in the amount of active agent (or combination of active agents) may or may not further decrease the blood pressure of these patients. The term encompasses patients that respond only insufficiently, i.e., exhibit some decrease in blood pressure, but short of the “predetermined increment” (to below 140/90 mm Hg). Generally, in those patients the amount of antihypertensive agent needs to be increased. But this may bring on or-aggravate side effects.

The terms “suboptimal” or “sub-threshold” amounts of active agent for monotherapy refer to amounts of active agent that insufficient to decrease blood pressure by the predetermined increment. “Suboptimal” or “sub-threshold” amounts may well vary from patient to patient. A patient who fails to achieve a decrease in blood pressure by the predetermined increment upon administration of a given dosage of active agent has either been administered a “suboptimal” or “sub-threshold” amount of active agent or may, alternatively, be a non-responder to the active agent. “Suboptimal” or “sub-threshold” amounts of active agent may be distinguished from the case of administration to a non-responder by increasing the administered dosage of active agent. In the case where a patient fails to achieve a decrease in blood pressure by the predetermined increment due to administration of a “suboptimal” or “sub-threshold” amount of active agent, administration of an increased dosage of active agent will cause the patient to achieve a decrease in blood pressure by the predetermined increment. In the case where a patient fails to achieve a decrease in blood pressure by the predetermined increment due to said patient being a non-responder, increasing the dosage of active agent will not cause the patient to achieve a decrease in blood pressure by the predetermined increment.

As used herein, the term “monotherapy” refers to the administration of a single active agent to treat hypertension.

The term “efficacy of treatment” refers to the potency of a drug in treating hypertension.

The term “in combination” refers to the concomitant administration of two (or more) active agents for the treatment of a single disease state. As used herein, the active agents may be combined and administered in a single dosage form, may be administered as separate dosage forms at the same time, or may be administered as separate dosage forms that are administered alternately or sequentially on the same or separate days. In one preferred embodiment of the present invention, the active agents are combined and administered in a single dosage form. In another preferred embodiment, the active agents are administered in separate dosage forms (e.g., wherein it is desirable to vary the amount of one but not the other). The single dosage form may include additional active agents for the treatment of the disease state. In a preferred embodiment, the single dosage form comprises lercanidipine and enalapril.

The term “combination therapy” refers to administration of at least two active ingredients “in combination” for the treatment of hypertension. In the present invention lercanidipine and enalapril may be further combined with one or more additional active ingredients, e.g., a diuretic and/or a β-receptor blocker and/or an angiotensin II receptor antagonist, without limitation.

In the present invention, the amount of enalapril administered to a patient in the combination therapy will be preferably within the range of 5 to 40 mg per day in a single or two divided doses. More preferably, the amount of enalapril will be 5-20 mg per day. The amount of lercanidipine will be preferably within the range of 5-40 mg, more preferably, 10-20 mg. The most preferred combinations are (i) 5 mg of enalapril and 5 mg of lercanidipine, (ii) 10 mg of enalapril and 5 mg of lercanidipine, (iii) 10 mg of enalapril and 10 mg of lercanidipine, (iv) 20 mg enalapril and 10 mg lercanidipine, and (v) 20 mg of enalapril and 20 mg of lercanidipine; but amounts may need to be optimized according to the needs of particular patient subpopulations depending on whether they are responders, partial reponders, nonresponders, or naive to lercanidipine and/or enalapril monotherapy at a tolerated dose (In the case of naive patients, the starting amounts of the combination may be even smaller that the indicated dose, e.g., 2.5 mg of enalapril).

### Pharmaceutical Compositions

The active agents of the combination of the present invention may be formulated into a single pharmaceutical composition or each can be administered in a different pharmaceutical composition. In any case, the pharmaceutical composition also may include optional additives, such as a pharmaceutically acceptable carrier or diluent, a flavorant, a sweetener, a preservative, a dye, a binder, a suspending agent, a dispersing agent, a colorant, a disintegrant, an excipient, a diluent, a lubricant, a plasticizer, an edible oil or any combination of two or more of the foregoing.

Suitable pharmaceutically acceptable carriers or diluents include, but are not limited to, ethanol; water; glycerol; aloe vera gel; allantoin; glycerin; vitamin A and E oils; mineral oil; PPG2 myristyl propionate; magnesium carbonate; potassium phosphate; vegetable oil; animal oil; and solketal.

Suitable binders include, but are not limited to, starch; gelatin; natural sugars, such as glucose, sucrose and lactose; corn sweeteners; natural and synthetic gums, such as acacia, tragacanth, vegetable gum, and sodium alginate; carboxymethylcellulose; polyethylene glycol; waxes; and the like.

Suitable disintegrators include, but are not limited to, starch such as corn starch, methyl cellulose, agar, bentonite, xanthan gum and the like.

Suitable lubricants include, but are not limited to, sodium oleate, sodium stearate, magnesium stearate, sodium benzoate, sodium acetate, sodium chloride and the like.

A suitable suspending agent is, but is not limited to, bentonite.

Suitable dispersing and suspending agents include, but are not limited to, synthetic and natural gums, such as vegetable gum, tragacanth, acacia, alginate, dextran, sodium carboxymethylcellulose, methylcellulose, polyvinyl-pyrrolidone and gelatin.

Suitable edible oils include, but are not limited to, cottonseed oil, sesame oil, coconut oil and peanut oil.

Examples of additional additives include, but are not limited to, sorbitol; talc; stearic acid; and dicalcium phosphate. Commercially available preparations containing enalapril (with or without another active ingredient) and lercanidipine can be used. Naturally, if the preparation contains more than one active ingredient, the amounts in the combination may have to be adjusted downwards.

### Unit Dosage Forms

The pharmaceutical composition may be formulated as unit dosage forms, such as tablets, pills, capsules, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. Unit dosage forms may be used for oral, parenteral, intranasal, sublingual or rectal administration, or for administration by inhalation or insufflation, transdermal patches, and a lyophilized composition. In general, any delivery of active ingredients that results in systemic availability of them can be used. Preferably the unit dosage form is an oral dosage form, most preferably a solid oral dosage, therefore the preferred dosage forms are tablets, pills, and capsules. However, parenteral preparations also are preferred.

Solid unit dosage forms may be prepared by mixing the active agents of the present invention with a pharmaceutically acceptable carrier and any other desired additives as described above. The mixture is typically mixed until a homogeneous mixture of the active agents of the present invention and the carrier and any other desired additives is formed, i.e., until the active agents are dispersed evenly throughout the composition. In this case, the compositions can be formed as dry or moist granules.

Tablets or pills can be coated or otherwise compounded to form a unit dosage form which has delayed and/or prolonged action, such as time release and sustained release unit dosage forms. For example, the tablet or pill can comprise an inner dosage and an outer dosage component, the latter being in the form of a layer or envelope over the former. The two components can be separated by an enteric layer which serves to resist disintegration in the stomach and permits the inner component to pass intact into the duodenum or to be delayed in release.

Biodegradable polymers for controlling the release of the active agents, include, but are not limited to, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

For liquid dosage forms, the active substances or their physiologically acceptable salts are brought into solution, suspension or emulsion, optionally with the usually employed substances such as solubilizers, emulsifiers or other auxiliaries. Solvents for the active combinations and the corresponding physiologically acceptable salts, can include water, physiological salt solutions or alcohols, e.g. ethanol, propane-diol or glycerol. Additionally, sugar solutions such as glucose or mannitol solutions may be used. A mixture of the various solvents mentioned may further be used in the present invention.

A transdermal dosage form also is contemplated by the present invention. Transdermal forms may be a diffusion-driven transdermal system (transdermal patch) using either a fluid reservoir or a drug-in-adhesive matrix system. Other transdermal dosage forms include, but are not limited to, topical gels, lotions, ointments, transmucosal systems and devices, and iontohoretic (electrical diffusion) delivery system. Transdermal dosage forms may be used for timed release and sustained release of the active agents of the present invention.

Pharmaceutical compositions and unit dosage forms of the present invention for administration parenterally, and in particular by injection, typically include a pharmaceutically acceptable carrier, as described above. A preferred liquid carrier is vegetable oil. Injection may be, for example, intravenous, epidural, intrathecal, intramuscular, intraruminal, intratracheal, or subcutaneous.

The active agents also can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active agents of the present invention may also be coupled with soluble polymers as targetable drug carriers. Such polymers include, but are not limited to, polyvinyl-pyrrolidone, pyran copolymer, polyhydroxypropylmethacryl-amidephenol, polyhydroxy-ethylaspartamidephenol, and polyethyl-eneoxideopolylysine substituted with palmitoyl residues.

lercanidipine can be formulated as a physiologically acceptable salt, e.g.,a salt with an inorganic or organic acid such as e.g. HCl, HBr, H₂SO₄, maleic acid, fumaric acid, tartaric acid and citric acid.

### Composition Examples

TABLE 1
Formulation I

| Ingredient | Amount (mg/tablet) |
|---|---|
| lercanidipine HCl | 10 |
| enalapril maleate | 10 |
| Lactose | 102 |
| Microcrystalline cellulose | 40 |
| Sodium bicarbonate | 8 |
| Sodium starch glycolate | 20 |
| Povidone K30 | 8 |
| Magnesium stearate | 2 |

A film coated tablet may be prepared using the cores described above and using the composition described in Table 2.

TABLE 2
Coating for tablet formulation shown in Table 1.

| Ingredient | Amount |
|---|---|
| Hypromellose | 1.91 mg |
| Talc | 0.15 mg |
| Titanium dioxide | 0.60 mg |
| Macrogol 6000 | 0.30 mg |
| Ferric oxide | 0.04 mg |

TABLE 3
Formulation II

| Ingredient | mg/tablet | mg/tablet |
|---|---|---|
| lercanidipine HCl | 5.0 mg | 10 mg |
| Lactose monohydrate | 35.0 mg | 30.0 mg |
| Microcrystalline cellulose | 39.0 mg | 39.0 mg |
| Sodium starch glycolate | 15.5 mg | 15.5 mg |
| Povidone | 4.5 mg | 4.5 mg |
| Magnesium stearate | 1.0 mg | 1.0 mg |
| Coating | | |
| Opadry OY-SR-6497 | | |
| Hypromellose | 1.91 mg | 1.91 mg |
| Talc | 0.15 mg | 0.15 mg |
| Titanium dioxide | 0.60 mg | 0.60 mg |
| Macrogol 6000 | 0.30 mg | 0.30 mg |
| Ferric oxide | 0.04 mg | 0.04 mg |
| Total | 103 mg | 103 mg |

### Administration

The pharmaceutical composition or unit dosage forms of the present invention may be administered by a variety of routes such as intravenous, intratracheal, subcutaneous, oral, parenteral, buccal, sublingual, opthalmic, pulmonary, transmucosal, transdermal, and intramuscular. Unit dosage forms also can be administered in intranasal form via topical use of suitable intranasal vehicles, or via transdermal routes, using those forms of transdermal skin patches known to those of ordinary skill in the art. Oral administration is preferred.

The pharmaceutical composition or unit dosage forms of the present invention may be administered to an animal, preferably a human being, in need of antihypertensive treatment. The pharmaceutical composition or unit dosage form of the present invention may be administered according to a dosage and administration regimen defined by routine testing in light of the guidelines given above in order to obtain optimal antihypertensive activity (especially for patients who are partial reponders or nonresponders to conventional monotherapy or to other combination therapies) and a decreased in blood pressure by the predetermined increment while minimizing toxicity or side-effects for a particular patient. However, such fine turning of the therapeutic regimen is routine in light of the guidelines given herein.

The dosage of the active agents of the present invention may vary according to a variety of factors such as underlying disease state, the individual's condition, weight, sex and age and the mode of administration. For oral administration, the pharmaceutical compositions can be provided in the form of scored or unscored solid unit dosage forms. For enalapril, the dosage forms comprise 5.0, 10.0, 20.0, or 40.0 mg for the symptomatic adjustment of the dosage to the patient to be treated. Preferably, the enalapril dosage forms comprise 5.0, 10.0, or 20.0 mg. For lercanidipine, the dosage forms comprise 5.0, 10.0, 20.0, or 40.0 mg for the symptomatic adjustment of the dosage to the patient to be treated. Preferably, the lercanidipine dosage forms comprise 10.0 or 29.0 mg.

For combination therapy according to the invention, the active agents may initially be provided as separate dosage forms until an optimum dosage combination and administration regimen is achieved. Therefore, the patient may be titrated to the appropriate dosages for his/her particular hypertensive condition. After the appropriate dosage of each of the active agents is determined to achieve a decrease of the blood pressure by the predetermined increment without untoward side effects, the patient then may be switched to a single dosage form containing the appropriate dosages of each of the active agents, or may continue with a dual dosage form. Preferably, the single dosage form comprises a first amount of enalapril from about 5 to about 40 mg per day. Preferably, the single dosage form also comprises a second amount of lercanidipine from about 5 to about 40 mg per day. In a preferred embodiment, the single dosage form comprises from about 5 to about 20 mg enalapril and from about 10 to about 20 mg lercanidipine. In a more preferred embodiment, the single dosage form comprises (i) 5 mg of enalapril and 5 mg of lercanidipine, or (ii) 10 mg of enalapril and 5 mg of lercanidipine, or (iii) 10 mg of enalapril and 10 mg of lercanidipine, (iv) 20 mg enalapril and 10 mg lercanidipine, or (v) 20 mg of enalapril and 20 mg of lercanidipine.

A pharmaceutical composition for parenteral administration contains from about 0.01% to about 100% by weight of the active agents of the present invention, based upon 100% weight of total pharmaceutical composition.

Generally, transdermal dosage forms contain from about 0.01% to about 100% by weight of the active agents, based upon 100% total weight of the dosage.

The exact dosage and administration regimen utilizing the combination therapy of the present invention is selected in accordance with a variety of factors including type, species, age, weight, sex and medical condition of the patient; the severity and etiology of the hypertension to be treated; the route of administration; the renal and hepatic function of the patient; the treatment history of the patient; and the responsiveness of the patient. Optimal precision in achieving concentrations of active agents within the range that yields efficacy without toxicity requires a regimen based on the kinetics of the drug's availability to target sites. This involves a consideration of the absorption, distribution, metabolism, excretion of a drug, and responsiveness of the patient to the dosage regimen. However, such fine tuning of the therapeutic regimen is routine in light of the guidelines given herein.

The pharmaceutical composition or unit dosage form may be administered in a single daily dose, or the total daily dosage may be administered in divided doses. In addition, co-administration or sequential administration of other active agents may be desirable. For example, addition of a diuretic, a preceptor blocker, or an angiotensin II receptor antagonist to the combination of lercanidipine and enalapril is contemplated by the present invention. The dosage amounts of the active agents may be adjusted when combined with other active agents to achieve desired effects (e.g., reduction of blood pressure by a predetermined increment, reduction or avoidance of a particular side-effect).

For combination treatment with both lercanidipine and enalapril, where the active agents are in separate dosage formulations, the active agents can be administered concurrently, or they each can be administered at separately staggered times. For example, enalapril may be administered in the morning and lercanidipine may be administered in the evening, or vice versa. Additional active agents also may be administered at specific intervals. The order of administration will depend upon the variety of factors including age, weight, sex and medical condition of the patient; the severity and etiology of the hypertension to be treated; the route of administration; the renal and hepatic function of the patient; the treatment history of the patient; and the responsiveness of the patient. Determination of the order of administration may be fine tuned and such fine tuning is routine in light of the guidelines given herein.

In a preferred embodiment of the present invention, the composition is administered daily to the patient. In a further embodiment, the composition of lercanidipine and enalapril is formulated into a single dosage form.

Patients that may be administered the composition described herein include, without limitation, partial responders or nonresponders to monotherapy with either enalapril or lercanidipine or with another calcium antagonist or ACE inhibitor and partial responders and nonresponders to other combination therapies. Another class of patients include responders to monotherapy that suffer from dosage-related side-effects, and responders to monotherapy who have been previously determined (or are expected to) become partial responders or nonresponders over time. The classification of patients into nonresponders, partial responders, and responders to a particular antihypertensive regimen is conventionally made by trial and error.

Recently, pharmacogenomic methods involving haplotyping have been utilized to identify responder patients, e.g., U.S. Pat. Nos. 6,200,754; 6,183,958; 6,110,684; and WO 98/45477.

### Uses-Methods for Treating Hypertension

The present invention contemplates a method of treating hypertension by administering to a patient a combination of lercanidipine and enalapril. In one preferred embodiment, the combination of the two active agents is formulated in one pharmaceutical composition. The patient is administered the combination at prescribed intervals (usually once daily) to maintain a physiologically effective amount of the active agents within the patient's system to produce the desired effect (i.e., a reduction of the patient's blood pressure by the predetermined increment). The composition may be administered by any route, as described above but oral administration is preferred for chronic treatment. The method may be used to treat hypertension in responders, partial responders, and nonresponders of monotherapy.

### EXAMPLES

The present invention will be better understood by reference to the following Examples, which are provided as exemplary of the invention, and not by way of limitation.

### Example I

### Model of Acute Angiotensin-Mediated Renal Hypertension in Anesthetized Rats

### Methods

Male Sprague-Dawley rats, weighing 250-300 g, were anaesthetized with pentobarbital sodium (35 mg/kg, i.p.) and placed on a thermic blanket. The temperature was maintained at 37° C. with a thermoregulator via a rectal probe. The animals were tracheotomized to facilitate spontaneous breathing. A polyethylene catheter was placed in the left jugular vein to allow for infusion of pentobarbital sodium to maintain anesthesia. The left femoral vein and artery were cannulated with polyethylene catheters to allow drugs administration and to monitor blood pressure, respectively.

The animals then underwent a left nephrectomy by excising the left kidney via a flank incision. The right kidney and renal vein, artery and ureter then were exposed via a right retroperitoneal incision, under a dissecting microscope. Silk threads were placed around both vessels and ureter. The cavity was then covered with Vaseline oil. See Recordati, et al. 2000, J. Hypertension, 18:1277-1287.

ECG (lead II) was recorded by means of subcutaneous electrodes. After 30-60 minutes of basal recordings of arterial blood pressure and heart rate the threads around the renal vessels and ureter were tied close to the renal hilum to induce complete renal ischemia of the right kidney. After 2 hours of ischemia (designed as “120” in Tables 1-4), the threads were removed to allow renal reperfusion and urine output. The reopening of the renal hilum and restoration of renal circulation, induced an increase in blood pressure that peaked 5-10 minutes and lasted about 60 minutes. Drugs (vehicle, enalapril (100 μg/kg), lercanidipine (10 μg/kg), or both lercanidipine (10 μg/kg) and enalapril (100 μg/kg)) were administered intravenously at 5 minutes after reperfusion began (defined as 125 in Tables 4-7).

Statistical analysis was performed during the reperfusion time (time 125-180 minutes). To evaluate the statistical differences among the treatment groups, data were analyzed using a three-way ANOVA (analysis of variance) with repeated measures on factor time and pre-planned multiple comparisons. Statistical analysis was performed by means of general linear model procedure (GLM) with SAS software version 6.12.

### Results

The effects of the different treatments studied are shown in Tables 4-10.

TABLE 4
Time course of the effects on SBP (systolic blood pressure),
DBP (diastolic blood pressure) and MBP (mean blood pressure)
after intravenous administration of vehicle (0.5 ml/kg) in
uninephrectomized anesthetized rats. Mean
value ± S.E.M. (n = 8).

| Time | SBP | DBP | MBP |
|---|---|---|---|
| min | mmHg | mmHg | mmHg |
| 0 | 108.1 ± 3.1 | 66.0 ± 2.0 | 80.0 ± 2.1 |
| Ischemia | | | |
| 120 | 111.5 ± 3.6 | 65.9 ± 2.8 | 81.1 ± 2.9 |
| Reperfusion | | | |
| 125 | 166.0 ± 9.4 | 119.8 ± 4.3 | 135.2 ± 5.8 |
| Drug | | | |
| 130 | 159.6 ± 8.1 | 115.8 ± 4.6 | 130.4 ± 5.5 |
| 135 | 152.0 ± 7.6 | 112.9 ± 5.4 | 125.9 ± 6.1 |
| 150 | 144.1 ± 7.6 | 106.1 ± 6.2 | 118.8 ± 6.6 |
| 165 | 131.9 ± 6.9 | 94.7 ± 7.0 | 107.1 ± 7.0 |
| 180 | 121.9 ± 7.6 | 84.8 ± 7.6 | 97.1 ± 7.5 |

TABLE 5
Time course of the effects on SBP (systolic blood pressure),
DBP (diastolic blood pressure) and MBP (mean blood pressure)
after intravenous administration of enalapril (100 μg/kg)
in uninephrectomized anesthetized rats. Mean
value ± S.E.M. (n = 8).

| Time | SBP | DBP | MBP |
|---|---|---|---|
| min | mmHg | mmHg | mmHg |
| 0 | 111.0 ± 2.6 | 69.6 ± 2.1 | 83.4 ± 2.1 |
| Ischemia | | | |
| 120 | 109.9 ± 5.1 | 65.1 ± 3.5 | 80.0 ± 3.9 |
| Reperfusion | | | |
| 125 | 176.8 ± 8.1 | 124.9 ± 4.3 | 142.2 ± 5.4 |
| Drug | | | |
| 130 | 146.1 ± 4.5 c | 109.9 ± 4.1 a | 122.0 ± 4.1 b |
| 135 | 143.9 ± 4.1 b | 107.1 ± 4.5 a | 119.4 ± 4.2 b |
| 150 | 135.4 ± 6.6 b | 95.9 ± 7.9 b | 109.0 ± 7.4 c |
| 165 | 128.5 ± 7.6 a | 86.5 ± 8.5 b | 100.5 ± 8.0 b |
| 180 | 123.4 ± 6.9 | 78.9 ± 7.6 a | 93.7 ± 7.2 a |

a = p < 0.05.
b = p < 0.01.
c = p < 0.001 vs the same time of vehicle

TABLE 6
Time course of the effects on SBP (systolic blood pressure),
DBP (diastolic blood pressure) and MBP (mean blood pressure)
after intravenous administration of lercanidipine
(10 μg/kg) in uninephrectomized anesthetized rats.
Mean value ± S.E.M. (n = 8).

| Time | SBP | DBP | MBP |
|---|---|---|---|
| min | mmHg | mmHg | mmHg |
| 0 | 110.4 ± 3.6 | 69.8 ± 2.5 | 83.3 ± 2.8 |
| Ischemia | | | |
| 120 | 114.5 ± 6.5 | 70.6 ± 5.4 | 85.3 ± 5.7 |
| Reperfusion | | | |
| 125 | 167.6 ± 8.9 | 119.3 ± 5.2 | 135.4 ± 6.4 |
| Drug | | | |
| 130 | 140.4 ± 3.8 b | 97.1 ± 3.1 c | 111.5 ± 3.2 c |
| 135 | 140.5 ± 3.7 a | 93.1 ± 4.0 c | 108.9 ± 3.7 c |
| 150 | 139.4 ± 3.7 | 89.5 ± 4.3 b | 106.1 ± 3.9 b |
| 165 | 132.0 ± 5.3 | 83.5 ± 4.6 a | 99.7 ± 4.7 |
| 180 | 128.1 ± 4.7 | 78.8 ± 4.0 | 95.2 ± 4.1 |

a = p < 0.05.
b = p < 0.01.
c = p < 0.001 vs the same time of vehicle

TABLE 7
Time course of the effects on SBP (systolic blood pressure),
DBP (diastolic blood pressure) and MBP (mean blood pressure)
after intravenous administration of lercanidipine
(10 μg/kg) and enalapril (100 μg/kg) in
uninephrectomized anesthetized rats.
Mean value ± S.E.M. (n = 8).

| Time | SBP | DBP | MBP |
|---|---|---|---|
| min | mmHg | mmHg | mmHg |
| 0 | 115.8 ± 3.2 | 74.3 ± 2.3 | 88.1 ± 2.5 |
| Ischemia | | | |
| 120 | 116.0 ± 4.8 | 73.3 ± 4.0 | 87.5 ± 4.3 |
| Reperfusion | | | |
| 125 | 172.5 ± 5.4 | 121.3 ± 3.2 | 138.3 ± 3.8 |
| Drug | | | |
| 130 | 124.3 ± 5.5 c | 79.9 ± 6.5 c | 94.7 ± 6.1 c |
| 135 | 125.4 ± 5.0 c | 76.6 ± 6.4 c | 92.9 ± 5.8 c |
| 150 | 123.4 ± 5.8 c | 72.6 ± 6.8 c | 89.5 ± 6.2 c |
| 165 | 118.1 ± 5.5 b | 66.3 ± 6.4 c | 83.6 ± 5.8 c |
| 180 | 113.4 ± 5.3 a | 62.6 ± 6.4 c | 79.5 ± 5.8 c |

a = p < 0.05.
b = p < 0.01.
c = p < 0.001 vs the same time of vehicle

TABLE 8
p values on systolic blood pressure

| | | | | lercanidipine + |
|---|---|---|---|---|
| Treatments | Vehicle | lercanidipine | enalapril | enalapril |
| Vehicle | — | | | |
| lercanidipine | p < 0.05 | — | | |
| enalapril | p < 0.05 | ns | — | |
| lercanidipine + | p < 0.001 | p < 0.001 | p < 0.001 | — |
| enalapril | | | | |

ns = not significant

TABLE 9
p values on diastolic blood pressure

| | | | | lercanidipine + |
|---|---|---|---|---|
| Treatments | Vehicle | lercanidipine | enalapril | enalapril |
| Vehicle | — | | | |
| lercanidipine | p < 0.001 | — | | |
| enalapril | p < 0.001 | p < 0.001 | — | |
| lercanidipine + | p < 0.001 | p < 0.001 | p < 0.001 | — |
| enalapril | | | | |

TABLE 10
p values on mean blood pressure

| | | | | lercanidipine + |
|---|---|---|---|---|
| Treatments | Vehicle | lercanidipine | enalapril | enalapril |
| Vehicle | — | | | |
| lercanidipine | p < 0.001 | — | | |
| enalapril | p < 0.01 | p < 0.001 | — | |
| lercanidipine + | p < 0.001 | p < 0.001 | p < 0.001 | — |
| enalapril | | | | |

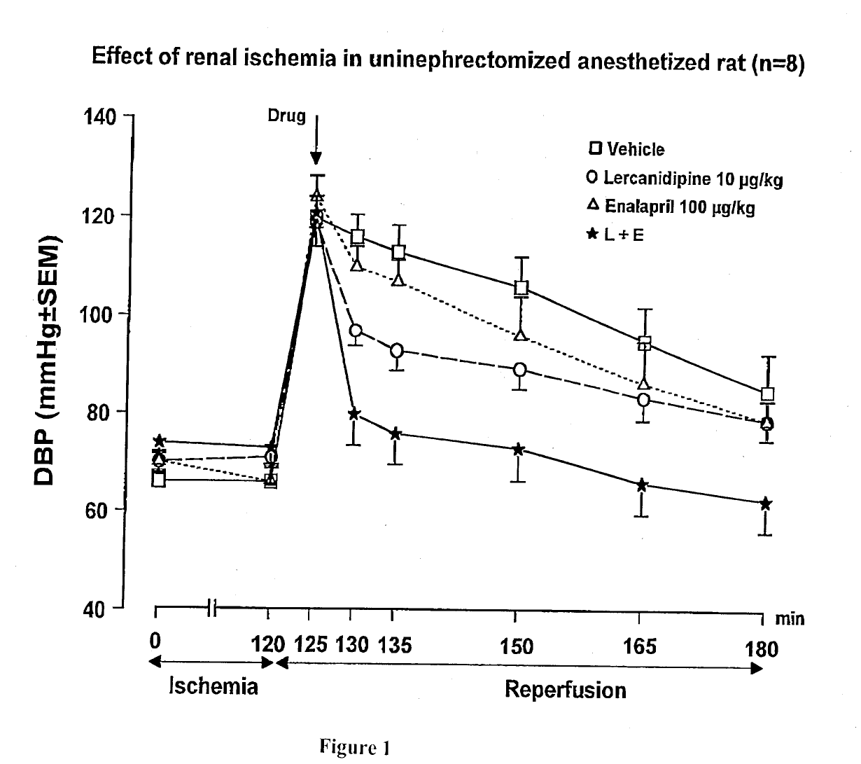

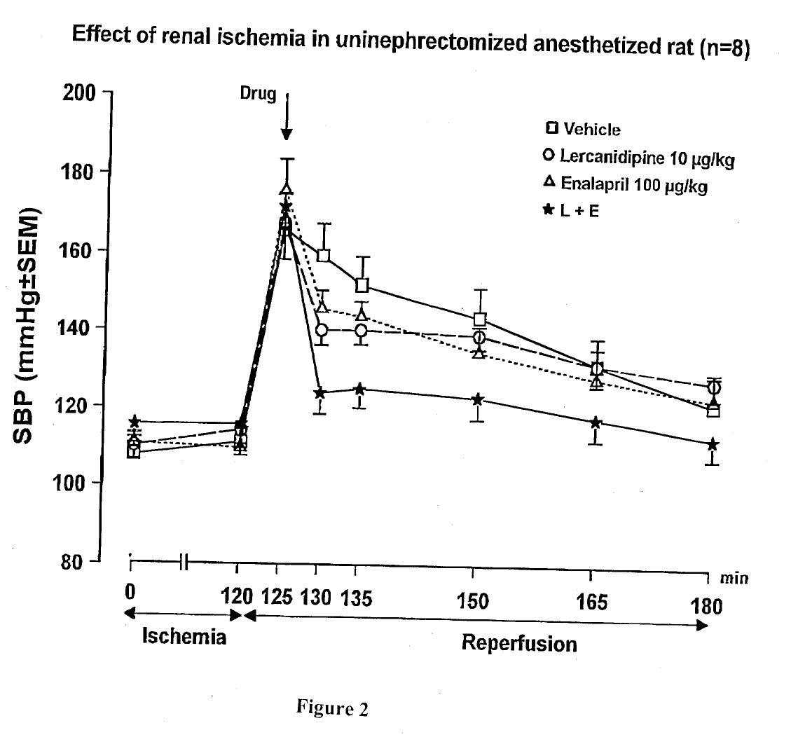

The foregoing experiments showed that lercanidipine and enalapril, when administered each alone, decreased blood pressure in a manner parallel to the spontaneous decrease of hypertension observed in the control (vehicle-treated) group. The overall decrease produced by lercanidipine and enalapril, separately, was statistically different (p<0.05 on SBP, p<0.001 in DBP, and p<0.001 on MBP) from vehicle-treated animals (See Tables 8-10). The antihypertensive effect of lercanidipine was greater than that produced by enalapril (See FIG. 1 and 2 and Tables 4-7) and this effect was statistically significant for diastolic blood pressure measurements (See Table 9).

When lercanidipine and enalapril were administered together, they significantly decreased the ischemia-induced hypertension (see FIGS. 1 and 2 and Tables 4-10). The combination of drugs produced an effect on blood pressure that was substantially more pronounced when compared to the effect on blood pressure produced by the individual drugs tested. The effect is synergistic, i.e., superadditive.

### Example II

### Association of Lercanidipine and Enalapril in Renal Hypertensive Dogs

### Methods

Male beagle dogs, 10-12 months old and weighing between 10-11 kg, were used. All dogs were trained for several weeks, to comply unstressfully to the test environment. Chronic sustained hypertension was induced in 4 dogs by bilateral renal artery constriction, according to the Goldblatt method “two-kidney, two clip hypertension”. Briefly, under sodium pentobarbital anaesthesia (35 mg/kg i.v.) during two different surgical interventions 15 days apart from each other, in sterile conditions, both renal arteries were clipped with original renal silver clips and narrowed by about 60-70%. After two months from the last intervention, an experimental renal hypertension was produced and the animals were used for the implantation of catheter.

Under sodium pentobarbital anaesthesia (35 mg/kg i.v.), in sterile conditions, the dogs were catheterized by inserting a sensor tipped catheter (Mikro-Tip) into the ascending aorta through the right femoral artery. The catheter was subcutaneously exteriorized at the back of the neck.

After a week recovery time from surgery, the animals were placed in a dog restrained unit, composed by a frame and a dog sling hammock, and connected to the pressure transducers in order to monitor the arterial blood pressure.

All the animals were alternatively treated with vehicle, lercanidipine, enalapril and the combination of lercanidipine and enalapril. The drugs were administered by oral gavage in a volume of 1 ml/kg at the following dosages:

(1) Vehicle (1 m/kg)

(2) lercanidipine (0.5 mg/kg)

(3) enalapril (3 mg/kg)

(4) lercanidipine 30 enalapril (0.5 mg/kg +3 mg/kg)

Blood pressure (systolic and diastolic) was monitored up to 6 h after the administration. Statistical analysis was performed from time 0 to time 360 min. To evaluate the statistical differences among the treatments, data were analyzed using a three-way ANOVA with repeated measures on factor time and pre-planned multiple comparisons. Statistical analysis was performed by means of GLM (general linear model procedure) with SAS software version 6.12.

### Results

The effects of different treatments studied are shown in Tables 11-16.

TABLE 11
Time course of the effects on SBP (systolic blood pressure)
and DBP (diastolic blood pressure) after oral administration
of vehicle (1 ml/kg) in conscious renal hypertensive dogs.
Mean value ± S.E.M. (n = 4).

| Time | SBP | DBP |
|---|---|---|
| min | mmHg | mmHg |
| Basal | 162.5 ± 4.8 | 124.8 ± 5.3 |
| 15 | 160.3 ± .5 | 123.5 ± 4.6 |
| 30 | 160.8 ± 5.1 | 123.5 ± 4.6 |
| 45 | 161.8 ± 5.2 | 122.5 ± 3.2 |
| 60 | 156.8 ± 4.5 | 121.0 ± 6.8 |
| 90 | 159.3 ± 5.3 | 120.3 ± 4.7 |
| 120 | 158.5 ± 7.2 | 124.0 ± 6.5 |
| 150 | 160.5 ± 8.7 | 124.0 ± 8.3 |
| 180 | 158.8 ± 7.2 | 124.3 ± 7.5 |
| 240 | 157.5 ± 4.3 | 122.0 ± 6.0 |
| 360 | 160.5 ± 6.5 | 125.3 ± 6.1 |

TABLE 12
Time course of the effects on SBP (systolic blood
pressure) and DBP (diastolic blood pressure), after oral
administration of lercanidipine (0.5 mg/kg) in conscious renal
hypertensive dogs. Mean value ± S.E.M. (n = 4).

| Time | SBP | DBP |
|---|---|---|
| min | mmHg | mmHg |
| Basal | 161.3 ± 3.8 | 117.5 ± 5.2 |
| 15 | 152.5 ± 6.6 | 107.0 ± 5.8 |
| 30 | 142.5 ± 4.8 b | 95.3 ± 3.6 c |
| 45 | 138.3 ± 5.1 c | 89.0 ± 4.8 c |
| 60 | 142.3 ± 7.4 a | 87.5 ± 6.3 c |
| 90 | 138.3 ± 5.7 c | 87.3 ± 5.3 c |
| 120 | 140.5 ± 5.9 b | 93.0 ± 5.6 c |
| 150 | 139.3 ± 10.1 c | 94.0 ± 7.2 c |
| 180 | 141.0 ± 7.2 b | 96.8 ± 5.1 c |
| 240 | 144.8 ± 6.8 a | 104.0 ± 5.0 a |
| 360 | 150.0 ± 6.8 | 108.0 ± 3.5 |

a = p < 0.05,
b = p < 0.01,
c = p < 0.001 vs the same time of vehicle

TABLE 13
Time course of the effects on SBP (systolic blood
pressure) and DBP (diastolic blood pressure), after oral
administration of enalapril (3 mg/kg) in conscious renal
hypertensive dogs. Mean value ± S.E.M. (n = 4).

| | | |
|---|---|---|
| Time | SBP | DBP |
| min | mmHg | mmHg |

| Basal | 164.8 ± 3.5 | 120.0 ± 4.2 |
|---|---|---|
| 15 | 162.8 ± 4.8 | 114.5 ± 4.9 |
| 30 | 160.3 ± 4.1 | 114.0 ± 4.7 |
| 45 | 158.0 ± 5.6 | 110.3 ± 4.7 |
| 60 | 158.0 ± 6.4 | 105.3 ± 5.6 a |
| 90 | 159.0 ± 5.8 | 107.5 ± 3.2 |
| 120 | 153.5 ± 7.5 | 105.0 ± 5.3 b |
| 150 | 158.5 ± 5.7 | 108.3 ± 4.5 a |
| 180 | 158.8 ± 5.5 | 110.5 ± 3.6 |
| 240 | 161.3 ± 7.2 | 116.0 ± 3.3 |
| 360 | 158.8 ± 5.9 | 111.8 ± 4.3 |

a = p < 0.05,
b = p < 0.01,
c = p < 0.001 vs the same time of vehicle

TABLE 14
Time course of the effects on SBP (systolic blood pressure)
and DBP (diastolic blood pressure), after oral
administration of lercanidipine + enalapril (0.5 mg/kg + 3
mg/kg) in conscious renal hypertensive dogs.
Mean value ± S.E.M. (n = 4).

| Time | SBP | DBP |
|---|---|---|
| min | mmHg | mmHg |
| Basal | 156.3 ± 4.7 | 119.5 ± 5.4 |
| 15 | 142.5 ± 5.2 a | 98.5 ± 1.2 b |
| 30 | 133.8 ± 9.0 c | 84.0 ± 5.9 c |
| 45 | 130.0 ± 9.4 c | 79.0 ± 6.6 c |
| 60 | 131.3 ± 9.0 c | 77.3 ± 5.5 c |
| 90 | 131.3 ± 6.6 c | 82.3 ± 6.6 c |
| 120 | 132.5 ± 8.3 c | 85.0 ± 6.5 c |
| 150 | 135.5 ± 6.1 c | 89.0 ± 4.1 c |
| 180 | 134.5 ± 8.3 c | 90.8 ± 5.4 c |
| 240 | 137.5 ± 6.6 b | 95.3 ± 4.5 c |
| 360 | 146.3 ± 7.5 | 108.8 ± 3.8 a |

a = p < 0.05,
b = p < 0.01,
c = p < 0.001 vs the same time of vehicle

TABLE 15
p values on systolic blood pressure

| | | | | lercanidipine + |
|---|---|---|---|---|
| Treatments | Vehicle | lercanidipine | enalapril | enalapril |
| Vehicle | — | | | |
| lercanidipine | p < 0.05 | — | | |
| enalapril | n.s | p < 0.001 | — | |
| lercanidipine + | p < 0.001 | p < 0.001 | p < 0.001 | — |
| enalapril | | | | |

ns = not significant

TABLE 16
p values on diastolic blood pressure

| | | | | lercanidipine + |
|---|---|---|---|---|
| Treatments | Vehicle | lercanidipine | enalapril | enalapril |
| Vehicle | — | | | |
| lercanidipine | p < 0.001 | — | | |
| enalapril | p < 0.001 | p < 0.001 | — | |
| lercanidipine + | p < 0.001 | p < 0.001 | p < 0.001 | — |
| enalapril | | | | |

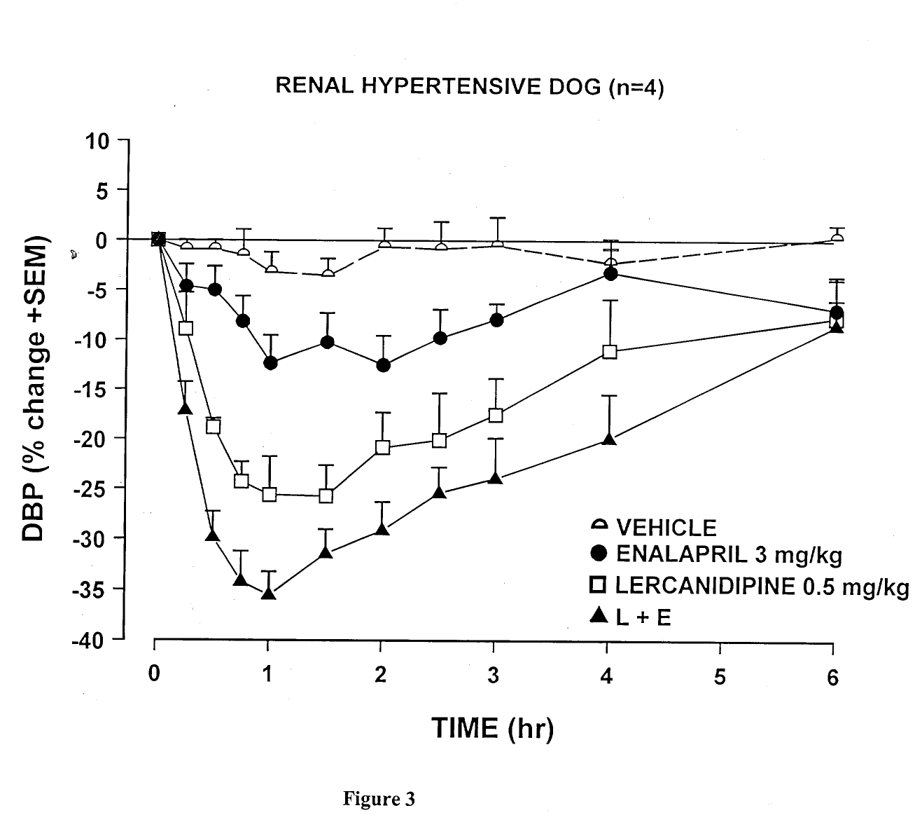

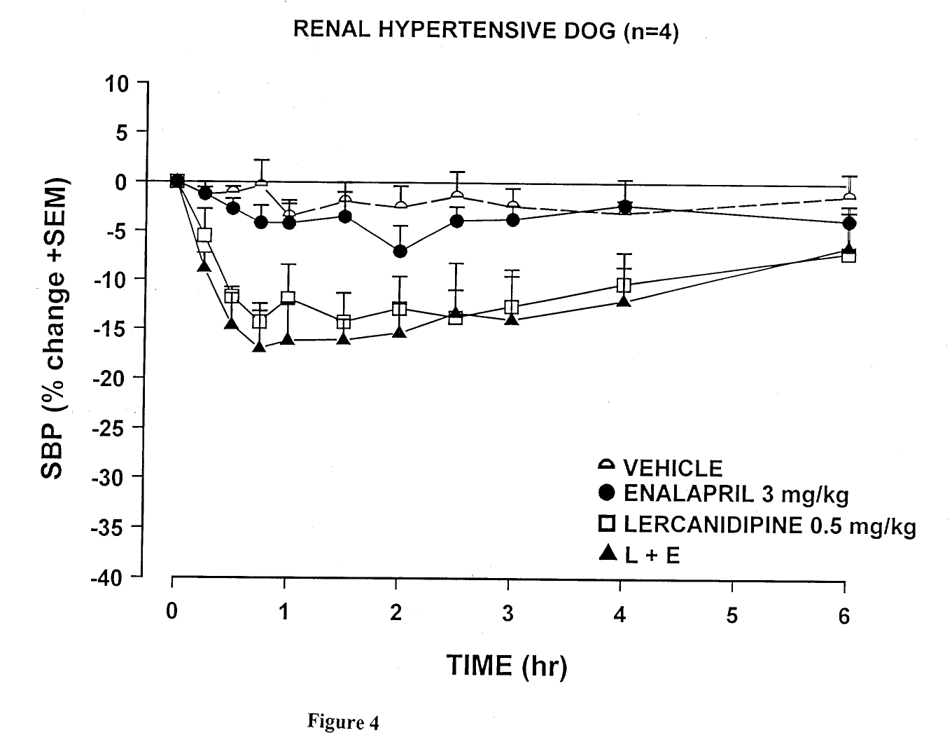

The results of this study demonstrate that the combination of lercanidipine and enalapril exerts a long lasting and additive (and at some time points synergistic) antihypertensive effect in an animal model of chronic renal hypertension (see FIGS. 3-4 and Tables 11-14). All the three treated groups induced an antihypertensive effect statistically significant from control group (Table 15-16). The reduction in blood pressure by the two drugs administered together is higher and statistically different on systolic and diastolic blood pressure than the two treatments alone. The above results indicate that the combination of lercanidipine and enalapril exerts a good control of blood pressure in hypertensive patients.

### Example III

### Clinical Study-Factorial Design

### Methods

Study Design

A multi-center, randomized, double-blind, placebo-controlled, parallel groups, factorial design trial was conducted. After a 14-day (±3 days) screening/washout period during which patients were washed from ongoing antihypertensive therapy, eligible patients with a sitting diastolic blood pressure (SDBP) of 95-109 mm Hg, entered the 28 day (±3 days) single-blind placebo run-in period during which they received placebo once-daily. Patients who were not responsive to this treatment and whose SDBP was 95-109 mm Hg, were randomized into an eight-week double-blind treatment phase. Baseline measurements were taken on Study Day 0 with post-therapy assessments during the double blind period scheduled on Study Days 14, 28, 42, and 56. Laboratory tests were conducted prior to the beginning of the screening period and on study Day 0 as well as at the end of the study.

Number Of Patients

The number of patients screened was greater than 800; 798 entered the 4-week single-blind placebo period, and 663 eligible patients were randomized into the 8-week double-blind treatment. The distribution of patients by group are shown in Table 17. The number in parenthesis represents the number of patients in each group available for efficacy evaluation.

TABLE 17
Patient Distribution in Study

| | | lercanidipine | lercanidipine | lercanipidine |
|---|---|---|---|---|
| | Placebo | (5 mg) | (10 mg) | (20 mg) |
| Placebo | 55 (54) | 54 (54) | 57 (57) | 54 (51) |
| enalapril (5 mg) | 55 (58) | 54 (54) | 52 (51) | 64 (63) |
| enalapril (10 mg) | 55 (53) | 52 (52) | 51 (51) | 58 (55) |

Study Drugs and Dosage and Duration of Treatment

The patients were randomized into one of the above noted 12 treatment groups. Tablets of lercanidipine (5 and 10 mg) and enalapril (2.5 mg) were encapsulated in the appropriate doses, in order to assure double-blind conditions. All tablets were of commercial origin except for the 5 mg lercanidipine, which were prepared in accordance with Table 3.

Patients were instructed to take one capsule of the study medication once daily 15 minutes before breakfast, between 6 and 10 am. All medications were to be withheld on mornings of clinic visits until after blood pressure evaluations at 22-26 hours following the previous day's dose of study medication.

Parameters Evaluated

Efficacy:

1. Trough (Minimum) (24±2 hours post-dose) sitting diastolic blood pressure (SDBP)

2. Trough (24±2 hours post-dose) sitting systolic blood pressure (SSBP)

3. Standing DBP and standing SBP—immediate and after 2 minutes

4. Percent of patients with SDBP<90 mm Hg

5. Percent of patients with SDBP<90 mm Hg or with SDBP 90 mm Hg but a decrease of≧10 mmHg

6. Percent of patients with SDBP<85 mm Hg

Safety:

1. Adverse events

2. Electrocardiogram

3. Laboratory tests

4. Physical exam

5. Heart rate

6. Changes in DBP and SDP from sitting to standing

Statistical Methods

The primary efficacy variable was the change from baseline to mean trough SDBP after 8 weeks of double-blind treatment. The analysis was performed in an “intent-to-treat” fashion, including all randomized patients who had received at least one dose of double-blind treatment, and had at least one post-baseline SDBP measurement 18-48 hours post-dose. In case of premature withdrawal from the study the last observation carried forward (LOCF) algorithm was applied (“end point analysis”).

The overall treatment comparison was performed using an analysis of covariance (ANCOVA) with treatment and centers as main effects and baseline value as covariate.

An additional ANOVA was performed using the factorial model in order to test for the interaction of lercanidipine dose and enalapril dose at each time-point. No interaction was found between lercanidipine and enalapril at the 0.10 level.

TABLE 18
Mean baseline values of SSBP and SDBP of patients within
each dosage group.

| | | lercanidipine | lercanipidine | lercanipidine |
|---|---|---|---|---|
| | Placebo | (5 mg) | (10 mg) | (20 mg) |
| Placebo | 157 ± 12/ | 155 ± 14/ | 157 ± 13/ | 154 ± 15/ |
| | 100 ± 4 | 100 ± 4 | 100 ± 4 | 100 ± 4 |
| enalapril | 159 ± 12/ | 156 ± 15/ | 157 ± 14/ | 158 ± 15/ |
| (5 mg) | 101 ± 4 | 100 ± 3 | 101 ± 4 | 100 ± 4 |
| enalapril | 153 ± 12/ | 158 ± 14/ | 157 ± 14/ | 155 ± 15/ |
| (10 mg) | 100 ± 4 | 101 ± 4 | 100 ± 4 | 100 ± 4 |

SDBP: Response Surface Analysis

The calculated changes in SDBP after treatment versus baseline SDBP are shown in Table 19.

TABLE 19
Change in SDBP

| | | lercanidipine | lercanidipine | lercanidipine |
|---|---|---|---|---|
| | Placebo | (5 mg) | (10 mg) | (20 mg) |
| Placebo | −6.6 | 7.4 (6.4-8.4) | −8.2 | −9.9* |
| | (5.4-7.8)^{a} | (7.3-9.2) | (8.6-11.2) | |
| enalapril | −7.7 | −8.5* | −9.3*,^{#} | −11.0*,^{#} |
| (5 mg) | (6.8-8.6) | (7.8-9.2) | (8.7-9.9) | (9.9-12.1) |
| enalapril | −8.8 | −9.6*,° | −10.4*,° | −12.0*,^{#} |
| (10 mg) | (7.6-9.98) | (8.6-10.6) | (9.5-11.4) | (10.8-13.4) |

^{a} Numbers in parentheses represents confidence intervals
Symbols indicate statistical significance versus placebo, enalapril alone at same dose, or lercanidipine alone at same dose:* p <0.05 versus placebo;
^{#}p < 0.05 versus enalapril;
^{°}p < 0.05 versus lercanidipine

These data show that patients treated with the composition exhibited a greater (and statistically significant) decrease in blood pressure when compared to patients that were given placebo. Patients treated with a combination composition where the combination contained 5 mg enalapril and at least 10 mg lercanidipine, exhibited a greater decrease than patients treated with enalapril alone. Patients treated with a combination composition where the combination contained 10 mg enalapril and 5 or 10 mg lercanidipine, exhibited a greater decrease in blood pressure than patients treated with lercanidipine alone.

Therapeutic Response Rates (SDBP<90 mm Hg)

The response rates (SDBP decreased below 90 mm Hg) for the patients within each dosage group are shown in Table 20. It should be noted that achievement of SDBP<90 mm Hg was not an aim of this study.

TABLE 20
Therapeutic response rates for study participants with
SDBP < 90 mm Hg.

| | | lercanidipine | lercanidipine | lercanidipine |
|---|---|---|---|---|
| | Placebo | (5 mg) | (10 mg) | (20 mg) |
| Placebo | 30% | 32% | 40% | 59%* |
| enalapril (5 mg) | 28% | 48%^{#,°} | 47%^{°} | 51%^{#,+} |
| enalapril (10 mg) | 43% | 44% | 55%* | 49%^{#} |

Symbols indicate statistical significance versus noted condition:
*p ≦ 0.01 versus placebo;
^{#}p < 0.05 versus placebo;
^{°}p < 0.05 versus enalapril (5 mg);
⁺p < 0.01 versus enalapril (5 mg).

These data show that patients treated with the composition exhibited a more pronounced decrease in blood pressure when compared to patients that were given placebo. Statistically significant decreases were observed with combinations that contained (i) 5 mg enalapril and at least 5 mg lercanidipine and (ii) 10 mg enalapril and at least 10 mg lercanidipine.

SSBP: Response Surface Analysis

The determined changes in SSBP after treatment versus baseline SSBP are shown in Table 21.

TABLE 21
Change in SSBP.

| | | lercanidipine | lercanidipine | lercanidipine |
|---|---|---|---|---|
| | Placebo | (5 mg) | (10 mg) | (20 mg) |
| Placebo | −8.1 (6.1-10.1)^{b} | −9.5 (7.8-11.1) | −10.8 (9.1-12.4) | −13.4* (11.1-15.6) |
| enalapril | −9.6 (8.1-11.2) | −11.0 (9.8-12.1) | −12.3* (11.2-13.3) | −14.9*^{,#} (13-16.7) |
| (5 mg) | | | | |
| enalapril | −11.1 (9.1-13.2) | −12.5* (10.7-14.2) | −13.8* (12.1-15.4) | −16.4*^{,#} (14.2-18.6) |
| (10 mg) | | | | |

^{b}Numbers in parentheses represents confidence intervals
*p < 0.05 versus placebo;
^{#}p < 0.05 versus enalapril

These data show that patients treated with the composition comprising 10 mg enalapril and at least 5 mg lercanidipine exhibited a greater (and statistically significant decrease) in blood pressure when compared to patients that were given placebo. Patients treated with a combination composition where the combination contained 5 mg enalapril and at least 10 mg lercanidipine, exhibited a greater decrease than patients with placebo.

Summary of Safety Results: Adverse Events

The number of patients with any adverse events related to study drug observed in the different groups is shown in Table 22. The number of patients who discontinued the study because of an adverse event is reported in parenthesis.

TABLE 22
Safety Results

| | | lercanidipine | lercanidipine | lercanidipine |
|---|---|---|---|---|
| | Placebo | (5 mg) | (10 mg) | (20 mg) |
| Placebo | 5 (1) | 8 (3) | 10 (1) | 10 (3) |
| enalapril (5 mg) | 9 (1) | 17 (0) | 10 (1) | 15 (6) |
| enalapril (10 mg) | 12 (1) | 8 (1) | 8 (1) | 13 (2) |

Safety analysis indicated that a lower number of patients dropped out for adverse events in the combination therapy groups than in the lercanidipine 20 mg group.

### Example IV

### Effect of Lercanidipine on Enalapril Treatment

### Methods

Study Design

This was a multicenter, randomized, double-blind, parallel-group study of the efficacy and safety of lercanidipine versus a comparative agent as an add-on therapy in patients with mild to moderate essential hypertension, uncontrolled on previous enalapril treatment.

The study was divided into three periods:

(1) During a lead-in period, naive patients were administered a stable dose of enalapril for 4 weeks. For patients already on a stable dose of enalapril for at least 2 weeks prior to study entry, the lead-in period was reduced to 2 weeks.

(2) The lead-in period was followed by a 2-week single-blind, run-in period where placebo was added to the current enalapril therapy to establish a baseline SDBP under stable enalapril therapy.

(3) After the run-in period, patients with a SDBP between 90 and 109 mm Hg and a SSBP between 140 and 179 mm Hg, inclusive, were administered 10 mg Leranidipine as add-on therapy for 8 weeks. For patients whose SBDP was≧85 mm Hg after 4 weeks of treatment, the dose of lercanidipine was increased to 20 mg.

Number of Patients

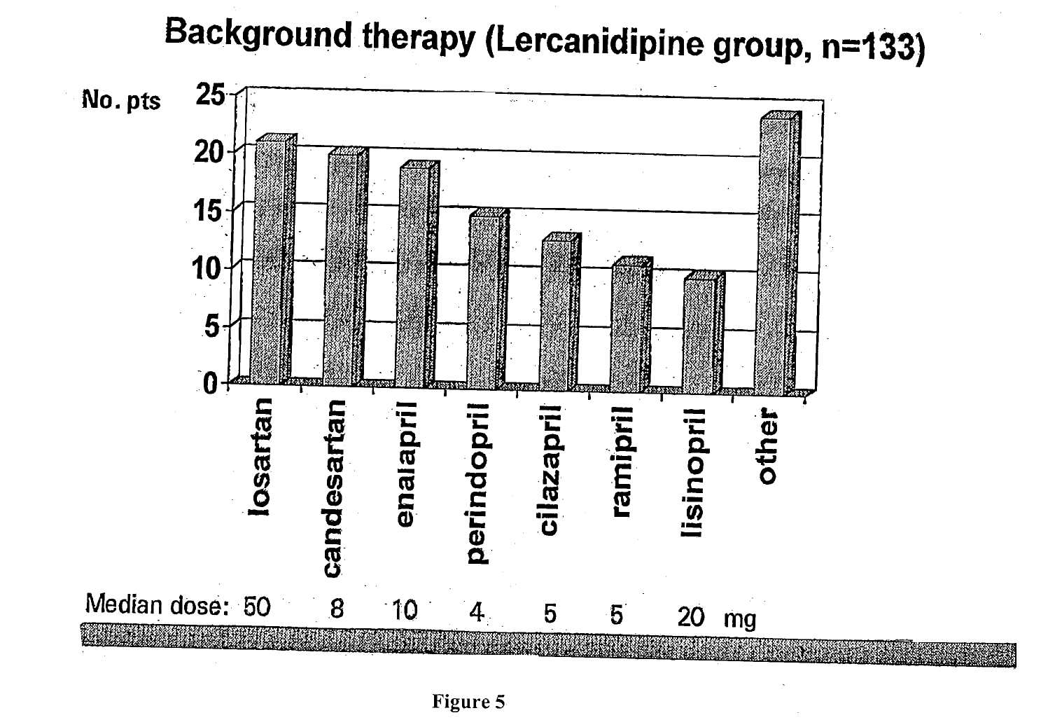

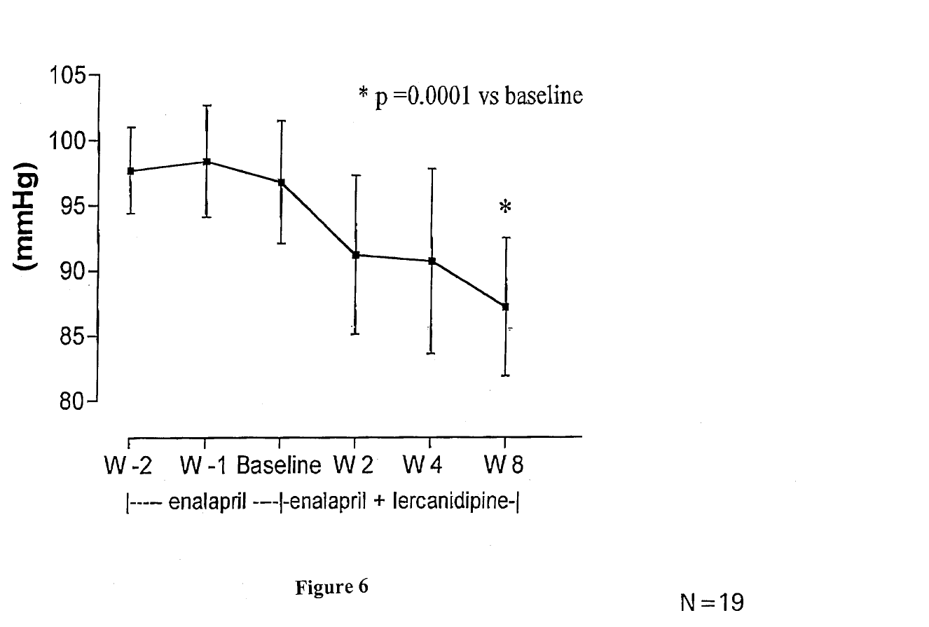

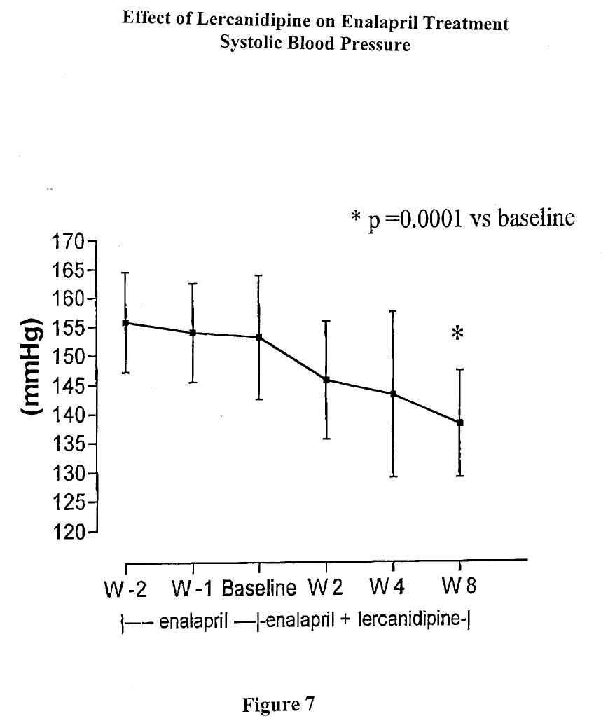

Nineteen patients, non-responders to a previous enalapril treatment (5 mg enalapril in 5 patients, 10 mg enalapril in 7 patients and 20 mg enalapril in 7 patients) were given lercanidipine (FIG. 5). All patients received 10 mg lercanidipine for the first 4 weeks (baseline to W4 in FIGS. 6 and 7) of treatment. After week 4, 13 of the 19 patients were administered an increased dosage of 20 mg lercanidipine.

Study Drugs and Dosage and Duration of Treatment

Patients were instructed to take medication once daily.

Parameters evaluated

Efficacy:

1. Trough (Minimum) (24±2 hours post-dose) SDBP

2. Trough (24±2 hours post-dose) SSBP

3. Percent of patients with SDBP<90 mm Hg after 4 and 8 weeks of treatment

4. Percent of patients with BP<140/90 mm Hg after 4 and 8 weeks of treatment

### Results

TABLE 23
Changes in SDBP and SSBP

| | Baseline | Week 2 | Week 4 | Week 8 |
|---|---|---|---|---|
| SDBP (mean ± | 96.8 ± 4.7 | 91.2 ± 6.1 | 90.7 ± 7.1 | 87.2 ± 5.3* |
| SD) | | | | |
| SSBP (mean ± | 153.1 ± 10.7 | 145.6 ± 10.2 | 143.1 ± 14.2 | 138.0 ± 9.1* |
| SD) | | | | |
| % patients with | ND | ND | 47% | 63% |
| SDBP <90 mm | | | | |
| Hg | | | | |
| % patients with | ND | ND | 32% | 42% |
| BP <140/90 | | | | |
| mm Hg | | | | |

*p = 0.0001 vs baseline (t-test)
ND = not determined

Addition of lercanidipine to enalapril therapy, for four weeks, decreased SDBP by an additional 6.1 mm Hg and SSBP by an additional 10 mm Hg. Additionally, 47% of the studied patients showed a DBP below 90 mm Hg and 32% of study participants achieved a blood pressure less than 140/90 (Table 23). As shown in FIGS. 6 and 7, eight week treatments, decreased SDBP by an additional 9.6 mm Hg and an additional SSBP by 15.1 mm Hg (p=0.0001). A SDBP of<90 mm Hg was observed in more than 60% of the patients after 8 weeks. A BP<140/90 mm Hg was observed in 42% of the patients after 8 weeks (Table 23).

### Conclusion

Clinical studies indicate that the predicted change versus baseline in the patients administered enalapril 10 mg and lercanidipine 10 mg in combination was significantly different from (1) placebo and (2) patients administered lercanidipine 10 mg monotherapy. Additionally, the observed effect in patients administered the combination of enalapril 10 mg and lercanidipine 10 mg was higher than that in patients administered enalapril 10 mg monotherapy. The effect observed in patients administered the combination of enalapril 10 mg and lercanidipine 10 mg was similar to the effect of lercanidipine 20 mg monotherapy (SDBP<90 mm Hg) patients.

Additional clinical studies further indicated that addition of 20 mg lercanidipine to existing enalapril therapy decreased SSBP greater than would be suggested when enalapril and lercanidipine were administered as monotherapies.

Together, these studies indicate that the effect associated with the combination of enalapril and lercanidipine is greater than the anti-hypertensive effect produced by the conventional dosages of either lercanidipine alone and enalapril alone. Moreover, there is a trend that the combination of lercanidipine and enalapril decreases the adverse events (i.e., side-effects) observed with lercanidipine monotherapy (at a higher dosage such as 20 mg).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Patents, patent applications, publications, procedures, and the like are cited throughout this application, the disclosures of which are incorporated herein by reference in their entireties.

What is claimed is:

1. A method for treating hypertension in a patient in need thereof, said method comprising administering to said patient a first amount of lercanidipine and a second amount of enalapril, wherein said amounts in combination are effective to reduce blood pressure in said patient by at least a predetermined increment; wherein each of said first amount and said second amount administered individually is ineffective to produce a reduction in blood pressure in said patient or said reduction in blood pressure is less than said predetermined increment, wherein said patient has not been previously determined to be unresponsive to enalapril.

2. The method as defined in claim 1, wherein said first amount is from about 5 -40 mg per day.

3. The method as defined in claim 2, wherein said first amount is from about 10-20 mg per day.

4. The method as defined in claim 3, wherein said second amount is from about 5-40 mg per day.

5. The method as defined in claim 4, wherein said second amount is from about 5-20 mg per day.

6. The method as defined in claim 5, wherein (i) said first amount is 5 mg per day and said second amount is 5 mg per day, (ii) said first amount is 5 mg per day and said second amount is 10 mg per day, (iii) said first amount is 10 mg per day and said second amount is 10 mg per day, (iv) said first amount is 10 mg per day and said second amount is 20 mg per day, or (v) said first amount is 20 mg per day and said second amount is 20 mg per day.

7. A method for treating hypertension in a nonresponder patient in need thereof, said method comprising administering to said patient a first amount of lercanidipine and a second amount of enalapril, wherein said amounts in combination are effective to reduce blood pressure in said patient by at least a predetermined increment, wherein said patient has been previously determined to be a nonresponder to monotherapy with lercanidipine or a nonresponder to combination therapy involving a different combination of antihypertensive agents.

8. The method as defined in claim 7, wherein said first amount is from about 5 -40 mg per day.

9. The method as defined in claim 8, wherein said first amount is from about 10-20 mg per day.

10. The method as defined in claim 9, wherein said second amount is from about 5-40 mg per day.

11. The method as defined in claim 10, wherein said second amount is from about 5-20 mg per day.

12. The method as defined in claim 11, wherein (i) said first amount is 5 mg per day and said second amount is 5 mg per day, (ii) said first amount is 5 mg per day and said second amount is 10 mg per day, (iii) said first amount is 10 mg per day and said second amount is 10 mg per day, (iv) said first amount is 10 mg per day and said second amount is 20 mg per day, or (v) said first amount is 20 mg per day and said second amount is 20 mg per day.

13. A method for treating hypertension in a partial responder patient in need thereof, said method comprising administering to said patient a first amount of lercanidipine and a second amount of enalapril, wherein said amounts in combination are effective to reduce blood pressure in said patient by at least a predetermined increment, wherein each of said first amount and said second amount administered individually is ineffective to produce a reduction in blood pressure by said predetermined increment.

14. The method as defined in claim 13, wherein said first amount is from about 5-40 mg per day.

15. The method as defined in claim 13, wherein said first amount is from about 10-20 mg per day.

16. The method as defined in claim 15, wherein said second amount is from about 5-40 mg per day.

17. The method as defined in claim 16, wherein said second amount is from about 5-20 mg per day.

18. The method as defined in claim 17, wherein (i) said first amount is 5 mg per day and said second amount is 5 mg per day, (ii) said first amount is 5 mg per day and said second amount is 10 mg per day, (iii) said first amount is 10 mg per day and said second amount is 10 mg per day, (iv) said first amount is 10 mg per day and said second amount is 20 mg per day, or (v) said first amount is 20 mg per day and said second amount is 20 mg per day.

19. A method for treating hypertension in a patient in need thereof, wherein said patient has been previously determined to be responsive to at least one of lercanidipine monotherapy or enalapril monotherapy but has ceased being adequately responsive thereto said method comprising administering to said patient a first amount of lercanidipine and a second amount of enalapril in combination, wherein said first amount and said second amount are in combination effective to reduce said patient's blood pressure by at least a predetermined increment.

20. The method as defined in claim 19, wherein said first amount is from about 5 -40 mg per day.

21. The method as defined in claim 20, wherein said first amount is from about 10-20 mg per day.

22. The method as defined in claim 21, wherein said second amount is from about 5-40 mg per day.

23. The method as defined in claim 22, wherein said second amount is from about 5-20 mg per day.

24. The method as defined in claim 23, wherein (i) said first amount is 5 mg per day and said second amount is 5 mg per day, (ii) said first amount is 5 mg per day and said second amount is 10 mg per day, (iii) said first amount is 10 mg per day and said second amount is 10 mg per day, (iv) said first amount is 10 mg per day and said second amount is 20 mg per day, or (v) said first amount is 20 mg per day and said second amount is 20 mg per day.

25. The method as defined in claim 1, wherein said enalapril and said lercanidipine are administered to said patient in a single dosage form.

26. The method as defined in claim 7, wherein said enalapril and said lercanidipine are administered to said patient in a single dosage form.

27. The method as defined in claim 13, wherein said enalapril and said lercanidipine are administered to said patient in a single dosage form.

28. The method as defined in claim 19, wherein said enalapril and said lercanidipine are administered to said patient in a single dosage form.

29. The method as defined in claim 1, wherein said enalapril and said lercanidipine are present in a composition.

30. The method as defined in claim 7, wherein said enalapril and said lercanidipine are present in a composition.

31. The method as defined in claim 13, wherein said enalapril and said lercanidipine are present in a composition.

32. The method as defined in claim 19, wherein said enalapril and said lercanidipine are present in a composition.

33. A pharmaceutical composition for treatment of hypertension comprising a first amount of lercanidipine and a second amount of enalapril, said first and second amounts in combination being effective to reduce blood pressure of a patient in need of treatment to within an acceptable value.

34. The composition of claim 33 wherein the first amount is within the range of 5-40 mg.

35. The composition of claim 33 wherein the second amount is within the range of 5-40 mg.

36. The composition of claim 35 wherein the first amount is within the range of 10-20 mg.

37. The composition of claim 35 wherein the second amount is within the range of 5-20 mg.

38. The composition of claim 33 wherein the lercanidipine and enalapril content of said composition is selected from the following pairs (i) through (v): (i) said first amount is 5 mg and said second amount is 5 mg per day, (ii) said first amount is 5 mg and said second amount is 10 mg per day, (iii) said first amount is 10 mg and said second amount is 10 mg per day, (iv) said first amount is 10 mg and said second amount is 20 mg, (v) said first amount is 20 mg and said second amount is 20 mg.

39. The method of claim 1 wherein said patient has not previously taken enalapril.

40. The method of claim 13 wherein said patient has not previously taken enalapril.
